(19) United States
(12) Patent Application Publication
(10) Pub. No.: US 2014/0163338 A1
(43) Pub. Date: Jun. 12, 2014

(54) Analyte Sensor with Slot Antenna

(71) Applicant: Bernd Roesicke, (US)

(72) Inventor: Bernd Roesicke, (US)

(73) Assignee: ROCHE DIAGNOSTICS OPERATIONS, INC., Indianapolis, IN (US)

(21) Appl. No.: 13/708,241

(22) Filed: Dec. 7, 2012

(51) Int. Cl. A61B 5/00

(52) U.S. Cl. CPC A61B 5/0031
USPC 600/309

(57) ABSTRACT

A medical device for detecting at least one analyte in a body fluid is disclosed. The medical device includes at least one implantable functional element and at least one driver having at least one electronic component. The functional element is connectable to the driver. The driver includes a housing having at least one metal housing and at least one wireless communication device. The metal housing has at least one slot structure. The communication device is adapted to communicate through the slot structure with an external apparatus.

This application is a continuation of International Application PCT/EP2011/059327, filed Jun. 7, 2011, which claims priority to EP Application No. 10 165 193.3, filed Jun. 8, 2010, both of which are hereby incorporated by reference in their entirety.

### BACKGROUND

The present disclosure relates to a medical device for carrying out at least one medical function on a human or animal body. In particular, the present disclosure relates to a sensor device for monitoring at least one body function, in particular for detecting at least one analyte in a body fluid. The present disclosure further relates to a method for producing a medical device for carrying out at least one medical function on a human or animal body, and in particular to a method for producing a sensor device for monitoring at least one body function, in particular for detecting at least one analyte in a body fluid, and in particular to a method for producing a medical device according to the present disclosure. Such medical devices, and in particular sensor devices, are generally used in medical therapy and diagnosis, for example in order to influence and/or monitor body functions. Examples are the continuous or discontinuous monitoring of analyte concentrations in at least one body fluid, for example interstitial fluid or blood. Envisionable analytes to be monitored are for example glucose, cholesterol, lactate, a metabolite in general or other types of analytes or analyte combinations. Essentially, however, the term “analyte” is intended to be interpreted in the broad sense and may refer to one or more chemical substances. In principle, the present disclosure is also generally applicable to other medical devices and apparatuses in the field of diagnosis, therapy or surgery since essentially, for example, at least one arbitrary body function may be recorded by means of the sensor device instead of one or more analytes and/or since, essentially, arbitrary physio-physical sensors, for example for blood pressure, temperature or movement, and/or actuators for influencing at least one body state, may be configured according to the disclosure.

The monitoring of particular body functions, in particular the monitoring of one or more concentrations of particular analytes, plays an essential role in the prevention and treatment of various diseases. Without restriction of further possible applications, the invention will be described below with reference to blood glucose monitoring. In principle, however, the present disclosure may be applied to other types of analytes and/or the monitoring and/or influencing of other types of body functions.

A method and a device for monitoring a hemodynamic status of a patient are known from U.S. Pat. No. 6,409,675 B1. Inter alia, a monitoring device is described. A use of a slot antenna is furthermore proposed.

A system for measuring an internal physiological parameter of a medical patient is known from US 2007/0167867 A1. Inter alia, an implantable sensor module is used. A telemetry signal in the form of an NIR signal is transmitted. The implant has a gold-plated electrode surface, which has a slot structure for avoiding eddy currents. In the slot structure, a central opening is provided which is without metallization and makes it possible to transmit the infrared signals.

U.S. Pat. No. 5,394,882 describes a wireless monitoring system having a first unit for detecting a movement of a patient and a second unit for receiving signals from the first unit. Inter alia, an exemplary embodiment of the first unit is described in which a disk-shaped slot antenna is used.

EP 2 187 555 A1 discloses a monitoring device for an analyte. Inter alia, a structure of corresponding sensor electronics is described.

US 2009/0182426 A1 discloses an implantable medical device. Inter alia, an antenna structure is described. In this case, it is disclosed that a metal housing is used, an antenna compartment made of a dielectric material extends externally around the housing, with an antenna which is embedded therein.

Besides so-called point measurements, in which a user takes a sample of a body fluid in a controlled way and it is analyzed for the analyte concentration, continuous measurements in the context of analyte monitoring are becoming increasingly established. For example, continuous glucose measurement in the interstitium (also referred to as continuous monitoring, CM) has recently become established as an important method for the management, monitoring and control e.g. of a diabetes status. At present, this continuous monitoring is in many cases restricted to Type I diabetics, i.e. diabetics who usually also wear an insulin pump. Since then, directly implantable electrochemical sensors have generally been employed, which are often also referred to as needle type sensors (NTS). In this case, the active sensor region is brought directly to the measurement site, which is generally arranged in the interstitial tissue, and glucose is converted e.g. by using an enzyme (for example glucose oxidase, GOD) into electrical charge which is related to the glucose concentration and can be used as a measurement quantity. Examples of such transcutaneous measurement systems are described in U.S. Pat. No. 6,360,888 B1 or in US 2008/0242962 A1.

Contemporary continuous monitoring systems are therefore generally transcutaneous systems. This means that the sensor per se is arranged below the user's skin. An evaluation and control part of the system (also referred to as a patch), however, is generally located outside the user's body, i.e. outside the human or animal body. The sensor is generally applied by means of an insertion set, which is likewise described by way of example in U.S. Pat. No. 6,360,888 B1. Other types of insertion sets are also known. The wearing time of a sensor is generally about one week. After this, influences, for example enzyme consumption and/or decapsulation in the body, generally make the sensitivity of the sensor decrease so that failure of the sensor is to be expected. Extension of the wearing time represents a contemporary field of development. This, however, means that the sensor and optionally components in direct connection therewith, for example an insertion needle, should be configured as replaceable components. Accordingly, the sensor and optionally further replaceable components generally constitute a so-called single-use part (disposable). The evaluation and control part of the system, on the other hand, is reused in most cases. Accordingly, this evaluation and control part is generally configured as a so-called reusable.

The separation into at least one disposable and at least one reusable, which is conventionally provided in the prior art, furthermore has the background that fully or partially implantable components for use in humans and/or animals need to be sterilized according to the standards in force. In the case of enzyme-based electrochemical glucose sensors, the enzyme embedded in the electrodes is in contact with the interstitium, i.e. the electrodes are open. Chemical or thermal sterilization is accordingly ruled out, since the enzyme of the electrodes would be damaged during this. As a general rule, only radiation sterilization can therefore be used. Electronic components, however, generally do not as a general rule withstand direct irradiation, for example with beta radiation, at the required radiation doses (usually 25 kGy). In the known devices, however, the effect of the separation into disposable and reusable is that only the disposable needs to be sterilized, while the reusable only has to be coupled to the disposable after sterilization.

Separation into a disposable and reusable, connected by a plug connection, does however have many disadvantages. For example, electrochemical glucose sensors usually operate according to a potentiostatic principle. In this case, the reference electrode used may in general only operate currentlessly. This, however, requires a very high-impedance configuration and good insulation of the entire potentiostat. During use, however, the system permanently lies either fully or partially (in particular the sensor) in a body tissue and therefore in an aqueous environment and/or is constantly exposed (in particular the reusable) to a very high relative air humidity. This promotes the formation of parasitic leakage resistances and/or leakage currents. The entire structure therefore needs to be sealed in a suitable way. In the case of a pluggable connection between the disposable and the reusable, however, in particular the plug connection constitutes a weak point of the insulation since parasitic leakage resistances and leakage currents can readily be formed in the region of the plug connection.

Full encapsulation of the disposable and of the reusable or elimination of the separation between a disposable and a reusable would accordingly be preferable. As described above, however, the drive electronics would then need to be protected in a suitable way against the ionizing radiation of the sterilization. From U.S. Pat. No. 6,565,509 B1, for example, an analyte monitor is known which comprises a sensor, a sensor control unit and a display unit. Inter glia, it is also proposed to obviate electrical contacts between the sensor and the sensor control unit. This arrangement, however, has the crucial disadvantage that damage to the controller can occur during radiation sterilization of the device. EP 1 178 841 B1, on the other hand, describes a method for protecting medical systems, which comprise sensitive semiconductor components, against high-energy radiation sterilization. It is proposed to accommodate the medical apparatus in a metallic protective housing, which is hermetically coupled to a carrier substrate that carries the sensitive semiconductor components. Such shieldings, however, in principle present numerous disadvantages in the case of the known systems. For example, in the case of the known electrochemical continuous glucose monitoring systems the sensor is located in the interstitium and the measurement value processing and storage part is located outside the body, directly on the skin but under the clothing. The continuously or discontinuously recorded glucose measurement values are buffered and transmitted on demand to an external apparatus, for example a data manager, a PDA (personal digital assistant), a PC or a mobile communication apparatus, for example by radio (radio frequency transmission, RF), where they are visually represented and/or processed further. Fully implanted sensors, for example analyte sensors, can also be operated via radio. As mentioned above, however, the present disclosure could in principle also be applied to other types of medical devices which comprise at least one implanted and/or implantable element, for example in general physio-physical sensors, for example invasive actuators such as cardiac pacemakers, insulin pumps, medicament dosing systems or the like. For reasons of hermetic insulation of the actual potentiostatic measurement system in the case of glucose sensors, communication via electromagnetic waves is particularly suitable. Optical systems are also suitable in principle for communication, but they have the disadvantage of line of sight with the receiver being necessary, which is not available particularly in the subcutaneous region or under clothing. However, antennas for receiving or emitting electromagnetic waves are a necessary component of radio systems. These, however, are generally formed by metallic structures in air. In this case, dipole antennas and/or half-wave dipole antennas in various embodiments may in particular be used for the present applications. Such anisotropic antennas act bidirectionally as transmission and reception antennas. Antennas can be adapted by shape and size to the specific frequency and tasks. If however a metallic protective housing is provided, as in EP 1 178 841 B1, then in general the antenna will also be fully shielded by a metallic shield. Because of this metallic shield, which acts as a Faraday cage, electromagnetic waves neither enter nor exit. Accordingly, the metallic shielding which is necessary for shielding the ionizing radiation during the sterilization has a detrimental effect on the communication properties of the sensor element. If on the other hand the shielding is removed again after the sterilization, this must be done under sterile conditions in order to avoid recontamination. Manufacture of electronic components under sterile conditions, however, entails considerable outlay.

Furthermore, a multiplicity of communication systems for medical and non-medical purposes are known in principle from the prior art. For instance, so-called “smart packages” are described in Christian Floerkemeier and Frank Siegemund: Improving the Effectiveness of Medical Treatment with Pervasive Computing Technologies, Workshop on Ubiquitous Computing for Pervasive Healthcare Applications at Ubicomp 2003, Seattle, Wash., October 2003. These contain monitoring of a medicament consumption in a medicament package and communication of a present consumption state via an electronic Bluetooth communication module to a mobile phone.

So-called “slot antennas” are also known in principle from other fields of the prior art, for example from aeronautical and astronautical engineering. For example, integration of slot antennas in the form of rectangular or circular split-rings into radio frequency circuits for WLAN communication is described in W. Ren: Compact Dual-Band Slot Antenna for 2.4/5 GHz WLAN Applications, Progress In Electromagnetics Research B, Vol. 8, 319-327, 2008.

It is accordingly desired to provide a medical device, in particular a sensor device, and a method for producing a medical device, in particular a sensor device, which avoid the above-described disadvantages of known medical devices. In particular, the medical device is intended to be sterilizable in a straightforward way by means of conventional sterilization methods, without thereby incurring damage to sensitive electronic components of the medical device. At the same time, however, the medical device is intended to be adapted to communicate wirelessly with other components.

### SUMMARY

The expressions “comprises”, “contains” or “has” and grammatical variants of these expressions, which are used below, are generally intended to mean that the components or elements introduced by these expressions may be contained exclusively, without other elements being present, or that one or more further components and/or elements may be contained besides the components or elements introduced by these expressions. For example, the expressions “A comprises B”, “A contains B” and “A has B” are intended to mean either that A exclusively contains B, that A is B or that A consists of B, or that A contains one or more further components and/or elements in addition to B.

A first aspect of the present disclosure provides a medical device for carrying out at least one medical function on a human or animal body. In the context of the present disclosure, a medical device is generally intended to mean a device which is adapted to carry out a medical function. A medical function is generally intended to mean a function which has a therapeutic and/or surgical and/or diagnostic effect. For example, the device may be adapted to influence and/or record at least one body function of the human or animal body. For example, this may be a physiological and/or physical state of the body. In order to record the body function, the device may for example be configured fully or partially as a sensor device and/or comprise a sensor device. In order to influence the body function, for example, the device may be configured fully or partially as an actuator and/or comprise at least one actuator, in which case the actuator may exert at least one stimulus on the body or a part of the body and/or it may influence the body in another way. The actuator may be adapted, for example, in order to exert a physical and/or chemical stimulus in the body. The actuator may for example exert an electrical stimulus on the body, for example with one or more stimulation electrodes. For example, the device may be configured fully or partially as a cardiac pacemaker, having at least one electrical actuator in the form of one or more stimulation electrodes. As an alternative or in addition, the device may also exert for example a chemical stimulus. For example, the device may be arranged fully or partially as a medication device and, for example, may have at least one actuator in the form of a medication pump and/or in the form of an active agent dispenser.

Without restriction of further possible configurations, the invention will be described below essentially with reference to medical devices, which are configured fully or partially as a sensor device or which comprise at least one sensor device. This sensor device may in general be configured for monitoring at least one body function and, optionally, adapted for the qualitative and/or quantitative detection of at least one analyte in a body fluid.

The monitoring of the body function may in principle be based on one or more physical and/or chemical and/or biological detection methods or measurement methods. For example, this may involve an electrochemical measurement and/or an optical measurement. For example, one or more analytes may be detected chemically, electrochemically or optically.

The term body function in the context of the present disclosure is in principle intended to be interpreted in the sense of one or more recordable properties and/or measurement qualities of a human or animal body. In particular, this may involve one or more properties which are characteristic of a state of health of the body. In particular, the body function may be at least one physiological function and/or at least one physiological characteristic of the body.

Examples of possible body functions, which may be recorded individually, in arbitrary combination or in combination with other body functions, are: a blood pressure; a pressure of at least one other body fluid and/or of at least one organ of the body; a heart rate; a respiratory rate; a temperature of the body and/or of a part of the body; a presence or absence or a concentration of one or more antibodies in at least one body fluid of the body, particularly in blood; a concentration of at least one analyte in at least one body fluid, in which case the concentration may be recorded qualitatively (presence or absence of the analyte) and/or quantitatively.

Without limitation to recording of further possible body functions, the disclosure will be described below essentially with reference to qualitative and/or quantitative detection of at least one analyte in at least one body fluid. With respect to the possible configurations of the analyte, reference may generally be made to the description above. In particular, the analyte may comprise at least one metabolite, for example glucose and/or lactate and/or cholesterol. The body fluid may in particular be selected from the group consisting of blood, interstitial fluid, saliva and urine. The sensor device may, in particular, be adapted for the continuous qualitative and/or quantitative detection of the at least one analyte. Accordingly, the sensor device may in particular be used in the scope of continuous monitoring, i.e. long-term monitoring over a time period of from several hours to several days, or even several weeks or months. Other configurations are, however also possible in principle.

The medical device comprises at least one implantable functional element. In particular, the sensor device may comprise at least one implantable sensor element. The medical device furthermore comprises at least one driver having at least one electronic component. The functional element may be fully or partially implantable. For example, an active part and/or a sensitive part of the functional element may be implanted into a body tissue of a user, while a supply lead may protrude out of the body tissue. Other configurations, for example fully implanted configurations, are however also possible in principle.

In the context of the present disclosure, a functional element is generally intended to mean an element which, in the medical device, can carry out the at least one medical function independently or in cooperation with other elements of the medical device. For example, it may be a sensitive element which can record the body function or, for example, can generate at least one signal which enables the body function to be deduced. For example, this may involve an electrical and/or optical signal. As an alternative or in addition, it may be an active element, in which case the active element is for example adapted to exert one or more of the stimuli mentioned above on the body or a part of the body, for example one or more electrical stimuli and/or one or more physiological stimuli, for example in the form of one or more medications.

In the context of the present disclosure, the term implantable is generally intended to mean that the functional element can be fully or partially introduced into a body tissue of the human or animal body. This introduction may, for example, be carried out transcutaneously or subcutaneously, for example in the scope of a surgical intervention. Accordingly, the term implantable implies that the functional element should firstly be dimensioned appropriately in order to be introduced into the body tissue. For example, the functional element, or for example an implantable part of the functional element, may be configured in such a way that it has a volume of not more than 3 cm³, preferably not more than 1 cm³. Furthermore, the functional element is intended to have biocompatible properties, at least on its surface. Thus, upon contact with body fluids and/or body tissue, the functional element should not dissolve and/or should not release any toxic substances, for example heavy metals. In order to achieve biocompatibility, appropriate passivation and/or coating may for example also be provided.

The sensor element may for example have at least one sensor chemistry which, in the presence of the at least one analyte, changes at least one detectable property, for example an electrochemically and/or optically detectable property. As described in detail below, it is particularly preferred for the implantable sensor element to be a sensor element for the electrochemical detection of the at least one analyte, for example a sensor element having at least two, preferably at least three electrodes, for example a working electrode and a reference and/or back electrode. Other configurations are, however, possible in principle.

The driver is used essentially to assist the functional element when carrying out the medical function and/or to cause the functional element to carry out the medical function. If for example the functional element exerts at least one stimulus on the body, then it is for example possible to provide a driver which, for example, specifies a point in time and/or an intensity and/or a duration of the exertion of the stimulus on the body. Optionally, the driver may also provide energy for the exertion of this stimulus. If the functional element is configured fully or partially as a sensor element, then the driver may for example record measurement values and/or signals delivered by the sensor element, and optionally provide them for evaluation. To this end, the evaluation may for example comprise measurement signal processing, in particular a so-called analog front end (AFE), and/or a memory part.

Furthermore, the driver may in general also comprise an energy supply. The at least one electronic component may in particular comprise at least one sensitive semiconductor component, for example an operational amplifier and/or another type of semiconductor component. The electronic component may furthermore comprise an application-specific integrated circuit (ASIC). In general, the electronic component may be an arbitrary electronic component which could suffer radiation damage during sterilization with ionizing radiation. In general, an electronic component is intended to mean an arbitrary component, in particular a semiconductor component, which can perform at least one electronic function, for example a rectifier function, an amplifier function, a transistor function, a memory function, a logical function, a function of a precision DC voltage source and/or DC current source, a clock function, a regulating function or another type of electronic function. Combined electronic components may also be used, i.e. components having more than one function.

The functional element, in particular the sensor element, is connectable to the driver. In the context of the present disclosure, connectable is generally intended to mean the possibility of establishing a connection between the elements connectable to one another, in particular an electrical and/or mechanical connection, for example by one or both of the elements connectable to one another comprising one or more connection elements, for example at least one plug connector or the like. The term connectable also includes the possibility that the elements are connectable to one another, here in particular the functional element (in particular the sensor element) and the driver, are already reversibly or even permanently connected to one another. As discussed in more detail below, the connection may be carried out by means of a fixed or permanent connection, i.e. by means of a connection which cannot be released by the user, at least not nondestructively. For example, this connection may be carried out by means of a cable or a part of the functional element, in particular of the sensor element, itself. The sensor element may, for example, be configured as a flexible sheet sensor element, at one end of which the electrodes of the sensor element are arranged and the other end of which is connectable, or even already connected, to the driver. Different configurations are possible.

The driver has a housing having at least one metal housing. This means that the driver is fully or partially enclosed by a housing which shields the driver against environmental influences, in particular against moisture. A housing is thus generally intended to mean an element which has a shielding effect at least against mechanical influences or chemical influences and has at least one fully or partially closed internal space, in which at least one element to be protected is accommodated. The housing in its turn has at least one metal housing, i.e. a housing which is fully or partially made of the at least one metallic material. The housing of the driver may be fully configured as a metal housing, or the metal housing may merely form a part of the overall housing. Different configurations are possible and will be described in more detail below. A metal housing is intended to mean a housing which is fully or partially made of at least one metallic material. The metal housing is in this case intended to be fully, predominantly or at least partially made of at least one metallic material and/or is preferably intended to comprise no nonmetallic components or only very few nonmetallic components, or it may also comprise at least one metallic material in addition to one or more nonmetallic components. For example, at least one metallic material may be introduced as a filler into at least one nonmetallic material, for example a plastic material. As an alternative or in addition, the metal housing may also comprise at least one layer structure having at least one nonmetallic layer and at least one layer of a metallic material. For example, a laminate structure may be used. For example, a plurality of properties may thus be improved or even optimized, for example sealing properties in relation to ingress of media, for example moisture, and shielding properties, for example against electromagnetic radiation and/or against ionizing radiation. For example, the metal housing may have a laminate structure in which one or more metal layers are combined with one or more plastic layers, for example by using a plastic layer, which serves as sealing and/or as corrosion protection and/or to increase the biocompatibility, as the outermost layer. The metal may for example comprise aluminum, copper, iron, lead or other metals or a combination (for example a mixture and/or an alloy and/or a layer structure having layers of different metals) thereof and/or other metals. For example, the metal housing may have a thickness which is at least 0.5 mm, optionally at least 1 mm or even at least 2 mm. The metal housing may in particular have a total thickness of between 0.5 mm and 10 mm, optionally a thickness of from 1 mm to 5 mm, for example a thickness of from 2 mm to 3 mm. The thickness may, for example, be dependent on the choice of material. If a plurality of layers are provided, then the individual layers may for example have a thickness of from 0.05 mm to 8 mm, for example from 0.1 mm to 5 mm and particularly preferably from 0.2 mm to 3 mm. With the structure, it is possible in particular to achieve a compromise between requirements in respect of the shielding thickness and the radiation attenuation on the one hand, and in terms of the requirements for minimal volume and/or minimal weight on the other hand.

The driver further has at least one wireless communication device. A wireless communication device is intended to mean a device which enables the driver to communicate unidirectionally or bidirectionally with apparatuses outside the medical device, in particular outside the sensor device. In particular, the communication device may be adapted to enable electromagnetic communication. For example, the driver may be adapted to record electrical signals from the sensor element and/or to buffer measurement values. Preprocessing or at least partial processing of these signals and/or measurement values may also be carried out in the driver. Via the wireless communication device, for example, it is then possible to carry out interchange of measurement values with other apparatuses, for example interchange of measurement values with external apparatuses, for example a data manager, a PDA, a mobile communication apparatus, a PC, a laptop or a network. As an alternative or in addition, control instructions may be received, for example by the driver receiving particular instructions from an external apparatus. A wireless communication device is in this case intended to mean a device which is adapted in order to interchange data and/or instructions wirelessly. For example, the wireless communication device may comprise a device for radio communication, i.e. communication via electromagnetic waves in the radio frequency range, for example in the gigahertz range. As an alternative or in addition, wireless communication may, for example, also be carried out via inductive and/or electrical coupling. In particular, the wireless communication may be carried out in such a way that it is not necessary to establish any DC connection. This is advantageous particularly in the case of electrochemical sensor devices having at least one invasive electrochemical sensor, in which the sensor electrodes preferably constitute the only DC connection to the sensor device, without there being any other DC connection to the sensor device.

In order to resolve the technical dilemma described above of at least a partial shielding of the driver by a metal housing, in particular shielding of the sensitive electronic component by the metal housing, together with the possibility of maintaining wireless communication connections in an emission direction through the metal housing, it is proposed to configure the metal housing with at least one slot structure. A slot structure is in this case intended to mean a structure having at least one slot in the metal housing, i.e. an elongate opening and/or interruption of the metal housing, which has a width and a length, the width being significantly less than its length. For example, the at least one slot may have a length which is less than its width by at least a factor of 3, in particular by at least a factor of 5 or even at least a factor of 10, or preferably even at least a factor of 20. The width of the slot is optionally less than 5 mm, particularly preferably 3 mm or less, or even merely 1 mm or less. An aspect ratio of the slot, as a ratio of width to length, may in particular be dependent on the frequencies used and/or an emission characteristic when, as discussed in detail below, the slot is used as a component of a slot antenna. The slot may be configured as a simple straight slot. As an alternative, the slot may also be configured to be curved, bent or angled, with one or more straight or curved sections. For example, a meander structure is possible. As an alternative or in addition, the slot structure may also be configured to be fully or partially circular, oval or spiral. Furthermore, a branched structure of the slot may also be provided, with one or more branches. The above-described condition for the slot geometry may then in particular relate to one or more or even all sections of the branched structure, it also being possible to provide one or more sections which do not fulfill the conditions mentioned, in particular one or more sections not formed as a slot, in addition to one or more sections formed as a slot. The at least one slot therefore preferably constitutes a break in the dielectric conditions of the metal housing, in particular when, as discussed in more detail below, the slot is a component of a slot antenna. Via this break, emission of an electromagnetic wave from a predetermined line structure can take place in this case, for example from the metal housing.

The communication device is adapted to communicate through the slot structure with at least one external apparatus. Communication through the slot structure may on the one hand be interpreted as meaning that the communication takes place through the slot structure, for example by signals being transmitted through the slot structure from the interior of the housing into an outer region or emitted directly from the slot structure, for example by at least one emitter being arranged in the slot structure. As an alternative or in addition, the term communication through the slot structure may also include the slot structure itself being involved in the communication, so that the communication takes place for example by means of the slot structure. Examples will be explained in more detail below.

The communication with the external apparatus is in general intended to take place entirely wirelessly, particularly in order to avoid DC coupling. As explained above, the housing is intended to comprise the at least one metal housing. In this case, the driver is preferably arranged at least partially in the metal housing, in particular fully. This may, for example, be achieved by arranging at least the electronic component, preferably at least one radiation-sensitive electronic semiconductor component, fully or partially in the metal housing.

For example, the metal housing may fully or partially enclose the driver. For example, the metal housing may be arranged with respect to the electronic component in such a way that, as seen from the electronic component, the metal housing covers at least one solid angle of at least 2π around the electronic component, preferably a solid angle of at least 2.5π, particularly preferably a solid angle of at least 3π and ideally a solid angle of at least 3.5π or even 4π.

As mentioned above, the sensor element may comprise at least one electrochemical sensor having at least two, preferably at least three sensor electrodes arranged in a body tissue of a user in the implanted state of the sensor element. The driver may in particular comprise at least one potentiostat and/or at least one primary amplifier, which is connectable or connected to the sensor electrodes. One or more of these components may in particular form a so-called analog front end (AFE). In the context of the present disclosure, a potentiostat is intended to mean an electronic regulating amplifier with which a potential of one of the electrodes can be regulated to a desired value. For example, the potentiostat may comprise a precision DC voltage source. For example, the potentiostat may be adapted to adjust a current between a working electrode of the sensor element and a back electrode of the sensor element, in such a way that the desired potential is achieved. A reference electrode, the potential of which is defined in the electrochemical series, may in this case be used as a reference point. A primary amplifier may in principle be interpreted as meaning an arbitrary amplifier, to which a signal from the sensor electrodes is applied directly or indirectly. In particular, it may be a high-impedance input stage in which case the amplifier may generally have a gain of more than 1, less than 1 or even equal to 1. The input stage may have an input resistance which is comparatively high, for example in the range of more than 100 kohm, for example more than 1 megaohm or even more than 1 gigaohm. The sensor device may in this case be adapted in particular in such a way that the potentiostat and/or the primary amplifier, which may be configured fully or partially as semiconductor components, can be arranged in the metal housing. As an alternative or in addition, however, further electronic components may be arranged inside the metal housing, for example memory components, operational amplifiers, transistors or other electronic components. In this case, an arrangement in the metal housing is generally intended to mean an arrangement in which the metal housing shields the accommodated components in at least one direction so that ionizing radiation, such as is used for the sterilization, cannot reach these components. For example, the metal housing may have a convex and a concave side, in which case the components may for example be arranged on the concave side of the metal housing. For example, the metal housing may form a metal shell shielding the driver in at least one direction. A metal shell is in this case intended to mean a shell structure which is open in one direction. The metal housing may in particular be coupled hermetically to a carrier element, in particular to a circuit board, the carrier element carrying the at least one electronic component of the driver. For example, the carrier element may be configured as a circuit carrier, in particular as a circuit board, on which the electronic component is applied, the metal housing being coupled to the circuit carrier, in particular to the circuit board. Hermetic coupling is in this case intended to mean coupling which prevents ingress of moisture into an intermediate space between the carrier element and the metal housing. This hermetic coupling may, for example, be carried out by potting and/or adhesive bonding. For example, the metal housing may shield the circuit board in one direction, for example in a direction which is used as an incidence direction during the sterilization.

The functional element, preferably the sensor element, in particular the sensor electrodes, may optionally be arranged fully or partially outside the housing, for example for implantation in the body tissue. The functional element, in particular the sensor element, may in principle for example be connectable or connected via at least one plug connection to the driver, for example releasably. It is, however, preferable for the functional element, in particular the sensor element, to be connectable or connected to the driver in a fixed fashion. This means, as mentioned above, that the functional element, in particular the sensor element, is connected to the driver preferably without a plug connection and cannot be separated nondestructively from the driver by a user. For example, the functional element, in particular the sensor element, may be wired to the driver in a fixed fashion. In this way, hermetic insulations can be formed and/or leakage currents and/or leakage resistances can be substantially minimized. The driver and the functional element, in particular the sensor element, may in particular be configured overall as a disposable or single-use part, for example for a Wearing time of from several weeks to several days.

As mentioned above, the communication device is adapted to communicate through the slot structure with at least one external apparatus. As likewise explained above, this communication may in particular be carried out in principle in an arbitrary way, in which the slot structure is involved in the communication. For example, at least one communication element of the communication device may be introduced into the slot structure and/or into at least one slot of the slot structure. For example, at least one coil, by which communication can be carried out with an external apparatus, may be introduced into the slot structure, transmission of electromagnetic waves and/or for example inductive coupling taking place through the slot structure or into the slot structure. As an alternative or in addition to communication via electromagnetic emission in the far field, i.e. at distances of more than two times the electromagnetic wavelength, inductive and/or magnetic and/or capacitive couplings by means of the at least one slot structure may for example therefore also be envisioned, for example by introducing one or more inductive and/or magnetic and/or capacitive coupling elements into the at least one slot structure in the metal housing, or bringing them into the vicinity thereof.

It is, however, particularly preferred for the slot structure to comprise at least one slot antenna. The communication device may in particular be adapted to carry out and/or enable wireless communication and/or electromagnetic communication, for example radio communication, by means of the slot antenna. This electromagnetic communication may in particular be such that electromagnetic waves, particularly in the scope of free-field emission according to the Maxwell field equations, come from the slot antenna and/or are received by the slot antenna. The communication device and the slot antenna may thus be adapted and cooperate in such a way that the slot antenna is used as an antenna for emission and/or reception of electromagnetic waves. The proposed medical device and in particular the sensor device, for example, therefore differ significantly in this configuration from the above-described U.S. Pat. No. 5,394,882, in which an opening in a metallization is merely provided, although it does not itself act as an antenna but is merely used as an entry window or exit window for radio frequency waves.

A slot antenna is intended to mean an interruption in a metallic structure, in this case an interruption of the metal housing, via which emission of electromagnetic waves can take place with suitable excitation. For example, this slot antenna may have a dipole or half-wave dipole structure. More complex geometries are, however, also possible in principle. While antennas as part of the communication device are generally formed by metal structures in air or on a carrier substrate, in metal shields such as the metal housing here, owing to their effect as a Faraday cage, electromagnetic waves usually neither enter nor exit. In principle, however, electromagnetic waves are emitted and/or received when, in an otherwise homogeneous structure, for example in this case the structure of the metal housing, a break in the dielectric and/or magnetic field conditions is induced. In the case of the slot antenna, which operates according to Babinet's principle, this is exploited by breaking through a metal structure by one or more slots of suitable length and/or suitable geometry. As mentioned above, a slot is defined as an opening in the metal structure, in this case in the metal housing, which has a high aspect ratio, i.e. a high ratio of length to width.

Slot antennas, such as are used in the context of the present disclosure for the medical device and in particular the sensor device, are known in principle from the prior art. Slot antennas are conventionally used as antennas in aircraft construction. Emission from waveguides having a slot structure is described, for example, in EP 1 263 086 A2. Slot antennas offer numerous significant advantages for the present application, and they are ideally suitable for achieving the goal described above and resolving the technical dilemma of known devices. For instance, on the one hand it is possible to keep the metal housing which provides shielding of sensitive semiconductor components against the sterilization radiation. In this way, for example, the functional element, in particular the sensor element, and the driver can be connectable or connected to one another in a fixed fashion and subsequently sterilized, which provides significant advantages in terms of production technology. Nevertheless, radiation damage to the semiconductor components is avoided. Furthermore, a plug connection between the functional element, in particular the sensor element, and the driver can also be obviated, which, as mentioned above, offers advantages in the structure and as a result of which the signal quality can be significantly improved. For example, hermetic sealing of the driver can thereby be achieved. Either the slot geometry of the slot antenna may be selected in such a way that ionizing radiation cannot enter the interior of the metal housing, or the slot geometry and the position of the slot may be selected in such a way that there are no sensitive semiconductor components below the slot antenna in the interior of the metal housing which the ionizing radiation of the sterilization radiation could reach through the slot antenna. The slot antenna may in turn comprise one or more slots, which may be open and/or filled with air or another gas, although they may preferably also be sealed, for example with a dielectric material. For example, the dielectric material may be provided as a solid material. In particular, the dielectric material may be or comprise at least one plastic material, for example an epoxy resin and/or a polyurethane. The dielectric material should have a relative permittivity, or dielectric constant, ∈ᵣ which is preferably close to one, for example a dielectric constant <5, in particular <3, preferably <2 or even <1.5. The slot is thus located merely in the metal housing and therefore constitutes an interruption of the otherwise homogeneous structure of the metal housing, filling the slot with the dielectric material influences the antenna properties of the slot antenna not at all or only slightly. By a suitable selection of the aspect ratio, it is furthermore essentially possible to prevent ionizing radiation from passing through the slot structure into the housing.

Slot antennas are also already known in principle from other fields of technology. For example, slot antennas are recently known from transponder technology. For example, WO 2007/048589 A1 describes a transponder chip module for a transponder having a slot antenna. The transponder chip module has a transponder chip and contact points electrically connected thereto, which are arranged on surfaces of the transponder chip module facing away from one another. Slot antennas are also known in principle from RFID technology and from packaging technology, in which case for example metallizations which are provided, in particular for moisture insulation in the case of medicament blisters or so-called smart packs, are provided with suitable slot structures which act as antennas. An example of a slot antenna structure suitable for RFID technology is described in WO 03/092116 A2. If an RFID chip is connected to such slot antennas, then, as described above, a transponder is obtained. So-called smart packages provided with such transponders can store and/or modify data or even operate sensors, for example temperature sensors, moisture sensors or the like.

The communication device may in particular comprise at least one excitation device, which is adapted to excite the slot antenna to emit electromagnetic waves. For the configuration of such excitation devices, reference may be made to known slot antennas or alternatively the above-described WO 2007/048589 A1. The excitation of the slot antenna may, for example, be carried out in a wide fashion or wirelessly. For example, a tuned electromagnetic circuit may be provided, which can excite the slot antenna to emit electromagnetic waves electrically conductively (for example via one, two or more conductors connecting the excitation device to the metal housing and/or the slot structure) or else wirelessly, for example by means of a primary emitter arranged in the metal housing. The excitation device may also in particular have at least one inductive element in order to shorten a slot length of a slot of the slot antenna to a length of not more than 10 cm. This inductive element may, for example, comprise a coil or another type of inductive element in a connection between the excitation device, and/or an oscillator of the excitation device, and the slot antenna. The slot structure may in particular have a slot with a slot length of not more than 10 cm, preferably not more than 5 cm and particularly preferably not more than 1 cm, preferably even less than 1 cm. If a slot antenna is used, then, as mentioned above, it may in principle have an arbitrary geometry. For example, linear geometries may be used, in particular dipole and/or half-wave dipole geometries. Other geometries are also possible in principle, for example branched geometries. It is also possible, for example, to use fractal geometries. Such fractal geometries may comprise a multiplicity of slots, for example a multiplicity of branched slots, which are arranged at an angle to one another and, for example, form a fractal pattern. Fractal geometries are known in principle from the field of printed i.e. non-slotted antennas, for example from EP 1 326 302 A2. Such fractal geometries may in principle also be used in the context of the present disclosure for the slot structure, and in particular for the slot antenna. As explained above, the slot structure may furthermore be at least partially sealed with a dielectric material. This means that the at least one slot may be fully or partially filled with a dielectric material, which seals the interior of the housing fully or partially. In particular, the dielectric material may be selected in such a way that it has a dielectric constant which (in its real part and/or its imaginary part) differs greatly from that of the material of the metal housing, for example by at least a factor of 1.5, preferably by at least a factor of 2 and particularly preferably even at least by a factor of 3. For example, the dielectric material may comprise at least one plastic material.

As mentioned above, the metal housing may in particular have a shielding effect for sterilizing radiation, in particular for ionizing radiation. In the context of a production method according to the disclosure for producing a medical device, in particular a sensor device, radiation sterilization may in particular be used, in particular by using ionizing particle radiation and/or ionizing electromagnetic radiation, for example selected from alpha radiation, beta radiation, gamma radiation, X-radiation and electron radiation. For instance, as mentioned above and as is also possible in the context of the present disclosure, in medical technology it is common to use electron beam sterilization. The electron beams usually have a beam energy of between 3 MeV and 12 MeV, a typical sterilization dose being 25 kGy. The metal housing of the medical device, in particular of the sensor device, may accordingly have a shielding effect which, for electron radiation between 3 and 12 MeV, is at least a factor of 2, preferably at least a factor of 5 and particularly preferably at least a factor of 10, or even a factor of 50. In this way, for example, a radiation dose of originally 25 kGy can be reduced to at most 12.5 kGy, preferably 5 kGy and particularly preferably at most 2.5 kGy, at most 1.25 kGy or even at most 0.5 kGy. The latter takes into account the fact that, for example, conventional CMOS electronics typically still do not suffer permanent damage or consequential damage therefrom with radiation doses of from 0.5 to 1 kGy. For example, the metal housing may have at least one metal selected from one of the following metals: aluminum; iron; lead; copper; a noble metal, in particular gold, silver, platinum; or an alloy. Combinations of the metals mentioned and/or other metals or elements may also be envisioned. For example, the metal housing may have a thickness which is at least 0.5 mm, optionally at least 1 mm or even at least 2 mm. As explained above, the metal housing need not enclose the entire driver, but is merely intended to shield radiation-sensitive semiconductor components in such a way that they are protected during radiation sterilization, preferably directional anisotropic radiation sterilization. For example, the metal housing may be configured as a half-shell.

As explained above, the proposed slot structure only insubstantially interrupts the shielding effect. For instance, the slot structure may in particular have at least one slot which is geometrically configured and/or arranged in such a way that ionizing radiation entering the housing through this slot during the radiation sterilization does not reach sensitive semiconductor components. For example, the slot structure may have at least one slot and the driver may have at least one circuit board, which may for example fully or partially be configured rigidly and/or flexibly, the electronic component, in particular at least one electronic semiconductor component, preferably all the electronic semiconductor components, being arranged on the circuit board outside a perpendicular projection of the slot onto the circuit board.

In general, for example, the slot structure may have at least one slot, in which case a border of the slot may span a plane. This spanning may, for example, be carried out so that the plane is defined in such a way that either all points of the border lie on the plane or a sum of the squares of the distances between all points of the border and the plane is minimal. In other words, the border of the slot itself may be configured in a planar fashion, or alternatively curved. The driver may have at least one carrier element, the electronic component being arranged on the carrier element outside a projection of the slot, the projection being a projection perpendicular to the plane onto the carrier element.

The carrier element need not necessarily be configured in a planar fashion. For example, the carrier element may comprise at least one planar circuit carrier, for example at least one circuit board. As an alternative or in addition, however, it is also possible to use non-planar circuit carriers, for example so-called three-dimensional circuit boards. Non-planar circuit carriers may, for example, be producible by means of injection molding methods and/or by means of so-called molded interconnect device (MID) technologies. The carrier element may be configured to be fully or partially different from the housing. For example, the carrier element as an independent component may be introduced fully or partially into the housing. As an alternative or in addition, however, the carrier element may be fully or partially connected to the housing or even formed integrally with the housing or parts thereof. In particular, the at least one electronic component may also be applied fully or partially directly on the housing.

Preferably, the medical device and in particular the sensor device are even configured in such a way that no electronic component is arranged within an angle of incidence of ±5°, in particular ±10° and particularly preferably ±20° in the housing. For example, the slot structure may in general have at least one slot, a border of the slot in the sense of the definition above spanning a plane, the electronic component being arranged outside a region which is formed as a sum of cones, cone vertices of the cones being points on the border of the slot, cone axes of the cones being perpendicular to the plane and the cones having a vertex angle of 10° (corresponding to a deviation of ±5° from a perpendicular projection), in particular 20° (corresponding to a deviation of ±10° from a perpendicular projection) and particularly preferably 40° (corresponding to a deviation of ±20° from a perpendicular projection).

This means that no radiation-sensitive semiconductor components are intended to be arranged below the slot in the driver. In addition, the ionizing radiation may be oriented during the radiation sterilization in such a way that, if at all, it enters a region of the housing in which no sensitive electronic components are arranged, in particular no semiconductor components. Furthermore, the slot structure may have at least one metallic shielding element protruding into the interior of the metal housing. This shielding element may for example comprise a collar, which protrudes into the interior of the metal housing. The effect of this shielding collar, or this shielding element, may be that although ionizing radiation may possibly enter through the opening of the slot structure, it nevertheless strikes the shielding element and is either absorbed there or deviated into a direction in which no sensitive semiconductor components are arranged. As an alternative or in addition, the at least one shielding element, or the at least one shielding collar, may also be configured as a radiation trap and/or comprise a radiation trap.

As explained above, the housing may in particular be configured to be moisture-tight. This may, for example, be achieved by corresponding sealing elements and/or corresponding potting. For example, as mentioned above, the metal housing may be hermetically coupled to a carrier element, in particular a circuit carrier and particularly preferably a circuit board, the carrier element carrying the electronic component. For example, the metal housing as a metal shell may be placed onto the carrier element and, for example, potted with the carrier element or sealed in another way, for example by adhesive bonding. The functional element, in particular the sensor element, is preferably connectable or connected to the driver in a fixed fashion. For example, at least one supply line of the functional element, in particular of the sensor element, may be fed through at least one sealing element into the housing and connectable or connected there to the driver. This at least one sealing element may, for example, comprise a lip seal and/or another type of elastomer seal and/or an adhesive bond. The driver may be fully or partially configured separately from the functional element, in particular the sensor element, although it may also be fully or partially connectable or connected to the functional element, in particular the sensor element, or formed integrally with the functional element, in particular the sensor element. For example, at least one substrate of the functional element, in particular of the sensor element, may also be used simultaneously as a substrate of the driver. For example, a flexible circuit board or flex-cable may be used, which may form the functional element, in particular the sensor element, or at least a part thereof and simultaneously the driver or a part thereof, for example a substrate of the driver. The flex-cable, or the flexible circuit board, may for example be provided outside the housing with at least one cover, for example at least one cover coating, in which case the cover may be formed separately from the optional sealing element and separately from the housing, although a preferably seamless transition between the cover and the housing may also be provided, in particular by the functional element, in particular the sensor element, and/or its cover merging directly and without an interrupting edge into the sealing element and/or the housing.

Another aspect of the present disclosure provides a method for producing a medical device, in particular a sensor device. In particular, it may be a medical device and particularly preferably a sensor device according to one or more of the configurations described above or below. Accordingly, with respect to possible configurations of the medical device, in particular the sensor device, reference may be made to the description above. Other configurations of the medical device, and in particular of the sensor device, are however also possible in principle.

In the proposed method, at least one implantable functional element, in particular at least one implantable sensor element, and at least one driver having at least one electronic component are provided. This may for example be done under sterile conditions, for example in a sterile room. Mounting, on the other hand, may preferably be carried out outside the sterile room. The functional element, in particular the sensor element, is connectable to the driver, which in turn implies the possibility that the connection of the functional element, in particular of the sensor element, to the driver is part of the method according to the disclosure. This method step of connecting the functional element, in particular the sensor element, to the driver may also preferably be carried out fully or partially under sterile conditions. The connection may, for example, be carried out by connecting two or more electrode supply leads for electrodes of the functional element, in particular of the sensor element, to corresponding components of the driver, for example a potentiostat and/or a primary amplifier. The driver is fully or partially shielded by a housing having at least one metal housing. This may, for example, be done by placing the housing and/or the metal housing after connection of the functional element, in particular of the sensor element, to the driver onto one or more components of the driver and/or applying them onto a carrier element of the driver, the driver being fully or partially enclosed. Other configurations are, however, also possible in principle. Subsequently, sealing may optionally be carried out in addition. The shielding of the driver by the housing may also be carried out under sterile conditions. Shielding is in this case intended to mean at least substantially moisture-tight compartmentalization of one or more components of the driver, in particular of the at least one electronic component. In addition, as mentioned above, at least partial shielding against ionizing radiation, in particular electron radiation and/or beta radiation, is carried out by the metal housing.

The driver has at least one wireless communication device. The metal housing has at least one slot structure, the communication device been adapted to communicate through the slot structure with at least one external apparatus. In particular, this may be done by introducing at least one slot, in particular at least one slot antenna, into the metal housing before or after production of the shielding. If a slot antenna is provided, then suitable coupling of the communication device to the slot antenna is preferably provided, for example by a corresponding conductive or wireless connection between an excitation device of the communication device and the slot antenna.

The radiation sterilization by the ionizing radiation may generally comprise radiation sterilization with electromagnetic radiation and/or particle radiation. To this end, for example, alpha, beta, gamma, X- or electron radiation may be used, no distinction being made below between electron radiation and beta radiation. Combinations of the types of radiation mentioned may also be used. The use of ionizing radiation has the advantage that, in particular, sensitive electrodes of the sensor element are not compromise, or comprise only insubstantially, by chemical influences. The radiation sterilization with ionizing radiation may, in particular, be carried out by using anisotropic ionizing radiation, for example by applying the ionizing radiation directionally onto the medical device, in particular the sensor device. Preferably, this incidence direction is selected in such a way that the metal housing is arranged between the radiation source and the at least one electronic component, in particular the at least one radiation-sensitive semiconductor component, during the application of radiation. When using a metal housing in the form of a shell or half-shell, this may for example be carried out by the shell or half-shell covering the at least one electronic component in the manner of a dome and shielding it against the radiation, while for example other regions of the medical device, in particular of the sensor device, are accessible to the radiation. The production of the module, or the medical device, and in particular the sensor device may in particular take place under non-sterile conditions, for example outside a sterile room. The radiation sterilization may then, for example, be carried out in a state in which the medical device, in particular the sensor device, is already packaged, for example in a blister pack. For example, the medical device, in particular the sensor device, may first be produced in a non-sterile fashion, and subsequently packaged for example in a blister pack or another germ-tight package, and subsequently sterilized in such a way that the sterilizing radiation passes through the package. The incidence direction of the ionizing radiation onto the housing may in particular be selected in such a way that ionizing radiation entering through the slot structure into the housing does not reach the at least one electronic component or at least sensitive electronic components of the driver, for example sensitive semiconductor components. An incidence direction may in this case be interpreted in particular as meaning a spatial direction from which the ionizing radiation strikes the housing.

In summary, the following embodiments are considered to be particularly preferred in the context of the present disclosure:

### Embodiment 1

Medical device for carrying out at least one medical function on a human or animal body, in particular sensor device for monitoring at least one body function, in particular for detecting at least one analyte in a body fluid, wherein the medical device comprises at least one implantable functional element, in particular at least one implantable sensor element, and at least one driver having at least one electronic component, wherein the functional element is connectable to the driver, wherein the driver has a housing having at least one metal housing, wherein the driver has at least one wireless communication device, wherein the metal housing has at least one slot structure, wherein the communication device is adapted to communicate through the slot structure with at least one external apparatus.

### Embodiment 2

Medical device according to the preceding embodiment, wherein the driver is arranged at least partially in the metal housing.

### Embodiment 3

Medical device according to one of the preceding embodiments, wherein the medical device comprises a sensor device, in particular a sensor device for monitoring at least one body function and/or a sensor device for detecting at least one analyte in a body fluid, and wherein the functional element comprises at least one implantable sensor element, in particular at least one implantable sensor element for detecting at least one analyte in a body fluid.

### Embodiment 4

Medical device for carrying out at least one medical function on a human or animal body, in particular sensor device for monitoring at least one body function, in particular according to one of the preceding embodiments, in particular for detecting at least one analyte in a body fluid, wherein the medical device comprises at least one implantable functional element, in particular at least one implantable sensor element, wherein the medical device furthermore comprises at least one driver having at least one electronic component, wherein the functional element is connectable to the driver, wherein the driver has a housing having at least one metal housing, wherein the metal housing is made entirely of a metallic material, wherein the driver is arranged at least partially in the metal housing, wherein the metal housing fully or partially encloses the driver, wherein the driver has at least one wireless communication device, wherein the wireless communication device comprises a device for communication selected from the group consisting of radio communication, communication by inductive coupling and communication via electrical coupling, wherein the metal housing has at least one slot structure, wherein the communication device is adapted to communicate through the slot structure with at least one external apparatus so that the communication takes place by means of the slot structure.

### Embodiment 5

Medical device according to the preceding embodiment, wherein the medical device comprises a sensor device, in particular a sensor device for monitoring at least one body function and/or a sensor device for detecting at least one analyte in a body fluid, and wherein the functional element comprises at least one implantable sensor element, in particular at least one implantable sensor element for detecting at least one analyte in a body fluid.

### Embodiment 6

Medical device according to one of the preceding embodiments, wherein the functional element, in particular the sensor element, comprises at least one electrochemical sensor having at least two sensor electrodes which are arranged in a body tissue in the implanted state of the functional element, wherein the driver has at least one potentiostat and/or at least one primary amplifier, which is connected to the sensor electrodes, wherein the potentiostat and/or the primary amplifier are arranged in the metal housing.

### Embodiment 7

Medical device according to one of the preceding embodiments, wherein the metal housing forms a metal shell shielding the driver in at least one direction.

### Embodiment 8

Medical device according to one of the preceding embodiments, wherein the metal housing is hermetically coupled to a carrier element, in particular a circuit board, wherein the carrier element carries the electronic component.

### Embodiment 9

Medical device according to one of the preceding embodiments, wherein the slot structure comprises at least one slot antenna.

### Embodiment 10

Medical device according to one of the preceding embodiments, wherein the communication device comprises at least one excitation device, wherein the excitation device is adapted to excite the slot antenna to emit electromagnetic waves.

### Embodiment 11

Medical device according to one of the preceding embodiments, wherein the slot structure is at least partially sealed by at least one dielectric material.

### Embodiment 12

Medical device according to one of the preceding embodiments, wherein the metal housing has a shielding function for electron radiation between 3 and 12 MeV of at least a factor of 2, preferably at least a factor of 5 and particularly preferably at least a factor of 10.

### Embodiment 13

Medical device according to one of the preceding embodiments, wherein the metal housing has at least one metal selected from one of the following metals: aluminum; iron; lead; copper; a noble metal; an alloy.

### Embodiment 14

Medical device according to one of the preceding embodiments, wherein the slot structure comprises at least one slot, wherein a border of the slot spans a plane, wherein the driver has at least one carrier element, wherein the electronic component is arranged on the carrier element outside a projection of the slot, wherein the projection is a projection perpendicular to the plane onto the carrier element.

### Embodiment 15

Medical device according to one of the preceding embodiments, wherein the carrier element is a circuit board.

### Embodiment 16

Medical device according to one of the preceding embodiments, wherein the slot structure has at least one metal shielding element, in particular a shielding collar, protruding into the interior of the metal housing.

### Embodiment 17

Medical device according to one of the preceding embodiments, wherein the functional element, in particular the sensor element, is connected to the driver in a fixed fashion, wherein at least one supply lead of the functional element is fed through at least one sealing element into the housing.

### Embodiment 16

Method for producing a medical device, in particular a sensor device, in particular a medical device according to one of the preceding embodiments, wherein at least one implantable functional element, in particular at least one implantable sensor element, and at least one driver having at least one electronic component are provided, wherein the functional element is connectable to the driver, wherein the driver is shielded by a housing having at least one metal housing, wherein the driver has at least one wireless communication device, wherein the metal housing has at least one slot structure, wherein the communication device is adapted in order to communicate through the slot structure with at least one external apparatus, wherein the medical device is sterilized with at least one ionizing radiation.

### Embodiment 17

Method for producing a sensor device, in particular according to one of the preceding embodiments relating to a sensor device, wherein at least one implantable sensor element and at least one driver having at least one electronic component are provided, wherein the sensor element is connectable to the driver, wherein the driver is shielded by a housing having at least one metal housing, wherein the metal housing is made entirely of a metallic material, wherein the driver is arranged at least partially in the metal housing, wherein the metal housing fully or partially encloses the driver, wherein the driver has at least one wireless communication device, wherein the wireless communication device comprises a device for communication selected from the group consisting of radio communication, communication by inductive coupling and communication via electrical coupling, wherein the metal housing has at least one slot structure, wherein the communication device is adapted in order to communicate through the slot structure with at least one external apparatus so that the communication takes place by means of the slot structure, wherein the sensor device is sterilized with at least one ionizing radiation.

### Embodiment 18

Method according to one of the two preceding embodiments, wherein an anisotropic ionizing radiation, in particular a beta radiation, is used, wherein an incidence direction of the ionizing radiation onto the housing is selected in such a way that ionizing radiation entering into the housing through the slot structure does not reach the electronic component.

The medical device proposed above, in particular the sensor device, and the method for producing a medical device, in particular a sensor device, have numerous advantages over known medical devices and production methods. In contrast to the prior art, according to which the contradictory technical aspects described above are not achieved, or are achieved only unsatisfactorily, according to the proposed disclosure it is possible to ensure optimal sealing and shielding while at the same time having the possibility of providing wireless communication between the driver and at least one external apparatus via the communication device, preferably by means of the slot antenna. At the same time, the possibility of using a metal housing has considerable advantages over conventional housings which are generally entirely made of plastic, including in terms of the leak tightness of the housing. Owing to their molecular structure, plastics generally exhibit relatively high moisture permeation, so that in the case of conventional plastic housings, water and water vapor can penetrate into the plastic housing after a relatively short time (generally within hours to days) and generally cause undesired parasitic leakage currents in the driver there. According to the prior art, accordingly, high expenditures are necessary for optimization of the seal, for example in the form of multilayered structures or deposits of drying agent arranged between them. Furthermore, plastic injection molding methods are often used. Wall thicknesses of less than 1 mm can generally be controlled only with difficulty. Conventional housings corresponding to the prior art, and in particular multilayered housings, however, are therefore also forward in general comparatively thick-walled and therefore large. This becomes all the more important since implanted or semi-implanted should be made small and lightweight as possible for appropriate wearing comfort.

The possibility according to the present disclosure of using metal housings eliminates these disadvantages in a simple and elegant way. Metals have the advantage of substantially lower permeation and, at the same time, they are capable of attenuating sterilization radiation. In this way, the driver, in particular a potentiostat and/or a primary amplifier, can be hermetically insulated for reasons of processing relatively small signals and high requirements for insulation from the environment, while nevertheless being arranged close to the functional element, in particular close to the sensor element. Since the metal housing offers shielding against ionizing radiation, however, the functional element, in particular the sensor element, can at the same time be radiation-sterilized since electronic components of the driver are protected by the metal housing. A system interface can therefore be obviated. This eliminates the above-described disadvantages of the interface being handled by a user and the associated disadvantages of reliable sealing. In particular by a fixed connection between the functional element, in particular the sensor element, and the driver, for example the potentiostat, the above-described sealing problems can reliably be avoided. By using the slot structure, in particular the slot antenna, however, the communication between the driver, out of the metal housing, can at the same time be maintained. The metal housing may enclose the driver fully or only partially, communication with at least one external apparatus through the at least one slot structure being possible as before.

Overall, various advantages can thus be combined by means of the solution according to the disclosure. For instance, on the one hand the low permeation of metals can be utilized, which improves the insulation of the driver. In this way, the signal qualities can be increased and the risk of signals affected by error can be significantly reduced. At the same time, the metal housing as a “metal cage” ensures good shielding during radiation sterilization, in particular by means of beta radiation. The slot structure, particularly in the form of one or more slot antennas, allows radio from almost fully closed metal housings. These advantages permit new product classes and in particular miniaturized medical devices, and particularly preferably miniaturized sensor devices, since for example critical interfaces between functional elements, in particular sensor elements, and reusables can be obviated. It is even possible to produce medical devices, in particular sensor devices, which can be configured as full implants, i.e. medical devices and in particular sensor devices in which both the functional element, in particular the sensor element, and the driver connected thereto can be fully implanted into a body tissue of the user. These full implants may comprise chemically reactive electrodes. The full implants can be sterilized without the risk of radiation damage.

### BRIEF DESCRIPTION OF THE DRAWINGS

Further details and features of the disclosure may be found in the following description of preferred exemplary embodiments, particularly in connection with the dependent claims. The respective features may be implemented on their own, or several may be implemented in combination with one another. The invention is not restricted to the exemplary embodiments. The exemplary embodiments are represented schematically in the figures. References which are the same in the individual figures refer to elements which are the same or functionally the same, or which correspond to one another in terms of their functions. In the drawings:

FIG. 1 shows a first exemplary embodiment of a sensor device according to the disclosure having a sensor element and driver which can be separated by plug connectors;

FIGS. 2A and 2B show an exemplary embodiment of a sensor device having a permanently connected sensor element and driver;

FIGS. 3A and 3B show an exemplary embodiment of a fully implanted sensor device having an external readout apparatus; and

FIG. 4 shows an exemplary embodiment of a sensor device having a flexible circuit board as a sensor carrier.

### DETAILED DESCRIPTION

FIG. 1 schematically represents a first exemplary embodiment of a sensor device 110 according to the disclosure for monitoring at least one body function, in particular for detecting at least one analyte in a body fluid. The sensor device 110 serves as an exemplary embodiment of a medical device 111 for carrying out at least one medical function on a human or animal body.

The sensor device 110 firstly corresponds in terms of structure substantially to the sensor device described in EP 1 972 269 A1, and comprises a disposable 112, i.e. a single-use part, and a reusable 114, i.e. a multi-use part. In the exemplary embodiment represented, the disposable 112 comprises a sensor element 116. The sensor element 116 generally represents an exemplary embodiment of an implantable functional element 117. The sensor element 116 may for example comprise a flexible carrier 118, for example a sheet carrier, and two, three or more electrodes 120 for an electrochemical analyte detection. The disposable 112 furthermore comprises an electronics part 122 having an energy supply, for example in the form of a battery 124, and a memory element 126, for example in the form of an EEPROM. The memory element 126 may, for example, comprise batch-specific data for the sensor element 116.

In the exemplary embodiment represented, the disposable 112 is connected via a plug connector 128 to the reusable 114. This plug connector 128 may, like optionally one or more further plug connectors of the sensor device 110, comprise at least one sealing element 129, which may be a component of the plug connector 128 and/or of a housing 142 and/or further components, or which may be formed fully or partially as a separate component. The reusable 114 may in particular comprise a component which is denoted by “AFE” in FIG. 1 (analog front end) and which may for example comprise a potentiostat 130 and/or a primary amplifier 132 having a high input impedance. The reusable 114 may furthermore comprise a data-processing device 134 which, for example, may be adapted for measurement data evaluation or at least preliminary measurement data evaluation. The data-processing device 134 is denoted in FIG. 1 as a microcontroller (μC). The data-processing device 134 may have a separate memory or, as an alternative or in addition, it may resort to the memory element 126 of the disposable 112.

In the exemplary embodiment represented, the reusable 114 furthermore comprises a communication device 136. This, in the exemplary embodiment represented, is configured as a radiofrequency (RF) communication device 136, for example as a radio chip.

According to the structure described so far of the exemplary embodiment according to FIG. 1, there is a separation between the reusable 114 and the disposable 112. Besides the sensor element 116, the disposable 112 may for example comprise a base plate 138 or another type of carrier element, which contains the battery 124 and the memory element 126, for example a memory chip. This base plate 138 with the memory element 126 and the battery, as well as the reusable 114, together form a driver 140 of the sensor device 110. This example shows that the driver 140 may also be configured in a plurality of parts. For the mode of operation of the sensor device 110 according to FIG. 1, reference may substantially be made to EP 1 972 269 A1. The disposable 112 is generally used only for one wearing cycle, for example for one week. The reusable 114 together with the disposable 112 provide the functioning patch of the sensor device 110. The reusable 114 is preferably adapted for multiple uses, for example for more than 50 cycles and/or for use during one year.

In order to avoid current transfer past the measurement system 130, 132 per se, for example past the AFE, in the sensor device 110 according to FIG. 1 as a general rule all electrical parts in the reusable 114 and in the disposable 112, apart from the electrodes 120 themselves (which are in contact with the body tissue, for example the interstitium, and generate the measurement signal) must be fully DC-isolated from the environment. Furthermore, particularly in the reusable 114, it is necessary to prevent leakage currents from flowing between the parts carrying the measurement current and/or parts at live potential. The insulation resistances within the entire electrical circuit must therefore in general be comparatively high, for example more than 1012 ohms or even more than 1013 ohms. To this end, it is necessary to prevent moisture due to ambient moisture (for example from water vapor to liquid water) from being able to reach these parts and, possibly together with contamination (for example ions), forming a conductive electrolyte there. To this end, in the present disclosure, the electronic modules of the disposable 112 and of the reusable 114, i.e. the driver 140, are accommodated in housings 142. These housings 142 are, for example, configured fully or partially as a metal housing 144. For example, metal housings 144 in the form of metal cups may be provided. These metal cups may for example be produced by conventional metal processing methods, for example stamping and/or deep drawing. The electronic components 126, 130, 132, 134 and 136, or parts of these electronic components, may be applied on one or more carrier elements 146, for example one or more circuit boards. The equipped circuit boards may be introduced into the metal housing 144. Sealing may additionally be carried out, for example by potting and/or adhesive bonding.

The sensor device 110 produced in this way according to FIG. 1 may then be radiation-sterilized as a whole. For example, beta radiation is generally used for the radiation sterilization. In this case, the range of the beta radiation in the metal housing is generally of interest for the shielding effect of the metal housing 144. Primarily, this depends on the density of the material. In general, for example, beta radiation between 3 MeV and 12 MeV will be used, typical radiation doses of 25 kGy being employed. Beta radiation with an intensity of 10 MeV, for example, penetrates about 6 mm into iron and about 19 mm in the case of aluminum. Accordingly, iron is to be preferred as the material for the metal housing 144. Also highly effective is copper, which at the same time also absorbs the secondary radiation (X-ray bremsstrahlung) occurring during the beta radiation. A copper shield with a thickness of 3 mm is sufficient in order to sufficiently attenuate the radiation at 25 kGy. Good compromises in terms of the shielding effect, processing and weight are generally provided by the materials aluminum and copper. In principle, however, it is also possible to use other materials and compounds of various materials. For example, an aluminum layer would have the function of attenuating beta radiation, while a second layer of a heavy metal, for example lead, would lead to attenuation of the bremsstrahlung. Furthermore, a thin shell of a suitable polymer could be provided, which provides the biocompatibility function since heavy metals are generally not biocompatible. These examples show that the metal housing 144 need not consist exclusively of one and the same material. For example, it is also generally possible to use metal housings having a plurality of materials, and metal housings which are constructed in multiple layers. In this case, metallic materials may also be combined with nonmetallic materials.

The exemplary embodiment of the sensor device 110 as represented in FIG. 1 is a so-called continuous monitoring sensor. In principle, however, with the present disclosure it is also possible to derive analyte sensors or physio-physical sensors as well as actuators and combinations of such devices.

In the exemplary embodiment represented in FIG. 1, as explained above, the reusable 114 and the disposable 112 are connected together by means of plug connectors 128, for example plug-in contacts. The plug connectors 128 are in this case preferably fed through the metal housing 144 by means of plastic sleeves. The surfaces of the penetrations are preferably configured to be small in relation to the total surface area of the metal cups, or metal housing 144. The plug-in region is likewise hermetically sealed, for example by means of releasable seals, in particular by means of O-rings. In this case, conventional plug systems may in particular be used, for example pogo contacts or blade contacts. Other configurations are also possible.

When using the metal housing 144, the above-described problem arises that it acts as a Faraday cage and in principle does not transmit signals from the communication device 136. On the other side of the metal housing 144, for example the metal cup, of the reusable 114 from the body, i.e. in the desired emission direction for radio signals, in the exemplary embodiment represented a slot structure 148 is therefore introduced. In the exemplary embodiment represented, this slot structure 148 acts in particular as a slot antenna 150 having an elongate slot 152, which in the exemplary embodiment represented is represented as an angled slot. Other slot geometries, however, are also possible in principle. Radiofrequency signals are applied by means of an excitation device 154 (not represented in detail in FIG. 1) of the communication device 136 to the slot antenna 150, and the latter is excited to emit radio waves outside the metal housing 144. The plan view according to FIG. 1 shows that the slot antenna 150, or the slot 152, is preferably arranged in such a way that it does not lie above sensitive electronic components, for example the components 130, 132, 134, 136. This reduces the likelihood that radiation entering through the slot 152 during radiation sterilization will damage these components. In principle, however, other positions of the slot antenna 148 are possible, for example positions closer to the body. These, however, are generally not optimal in terms of their emission and/or reception characteristic. In order to obtain the best possible reception and/or emission characteristic, various slot structures 148 are furthermore possible. The geometry and configuration of these slot structures 148 may in this case be optimized so that, for example, spirals, meanders or similar structures may be used. Furthermore, by means of suitable slot structures, dimensions of the antenna structure can be matched to the frequencies and/or wavelengths used. In particular, the antenna may be shortened in this way. To this end, the excitation device 154 and/or the slot structure 148 itself may for example comprise one or more inductances. Owing to the requirement for compactness of such a system, high frequencies are generally more suitable than low frequencies because of the shorter wavelengths. The widespread ISM frequency of 2.4 GHz, which is freely available worldwide, leads to slot structures with a length of only a few cm. This can be shortened even further by suitable inductances. In principle, however, other wavelengths are also possible.

The slot 152 of the slot antenna 148 may furthermore be filled with an insulating and/or sealing compound. For example, this may be a polymer compound having a dielectric constant differing greatly from the metal of the metal housing 144. The lower permeation of the overall housing is thereby influenced not at all or only insubstantially, since the permeation is a function inter alia of the surface area.

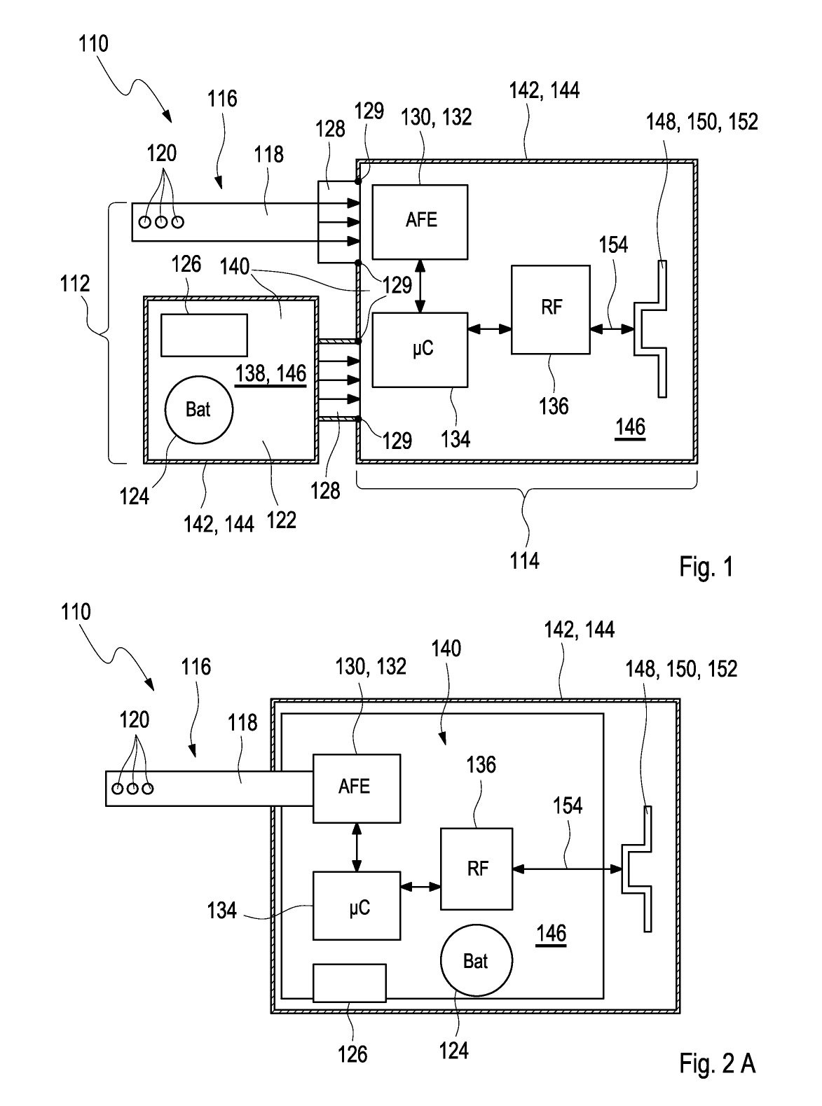

Since the sensor device 110 shown in FIG. 1 can now also be readily sterilized, FIGS. 2A and 2B show a second exemplary embodiment of a sensor device 110 as an example of a medical device 111, in which the plug connectors 128 and the separation of the sensor device 110 into a disposable 112 and a reusable 114 are obviated. In this exemplary embodiment, the sensor element 116, as an example of a functional element 117, is connected in a fixed fashion to integrate with the driver 140, and there in particular the AFE 130, 132. In this case, miniaturization and/or combination of a plurality of electronic components of the driver 140 may be carried out, for example by combining a plurality of the components represented, or even all of the components represented, to form a common integrated circuit and/or integrated semiconductor component, for example an ASIC (user-specific integrated circuit). This, for example, can contribute to increasing the economic viability of the integrated sensor device 110, even though there is no longer any reusable part of the sensor device 114. Also, for example, sensor working lives in the interstitium can be extended beyond one week, and the electronics of the driver 140 can overall be rendered highly economical.

FIG. 2B shows a sectional representation through the sensor device 110 according to FIG. 2A from the side. A body tissue 156, into which the sensor element 116 is partially implanted with the side of its electrodes 120, is also symbolically represented. It can be seen that the metal housing 144 in the exemplary embodiment represented constitutes a half-shell which is placed over the carrier element 146 that carries all or at least some of the electronics components 130 to 136. The half-shell is thus for example arranged precisely in relation to the communication device 136 in such a way that the latter is arranged in a possible emission direction for communication signals of radio communication. The half-shell may then be hermetically closed off from the body tissue 146 by one or more seals 158, for example by adhesive bonding and/or potting, so that the carrier element 146 with the electronic components can be arranged in the interior of a hermetically shielded housing 142. The carrier 118 of the sensor element 116, with supply lines correspondingly accommodated thereon, may then be fed into the interior of the housing 142 through one or more sealing elements 160 (not represented in detail in FIG. 2B).

Furthermore, it can also be seen from FIG. 2B that the slot structure 148 is arranged remotely from the electronics components 130 to 136. Radiation sterilization 162 is symbolically indicated in FIG. 2B, although it is naturally carried out not with the sensor device 110 applied on the body tissue 156 but already during production and before use by a user. The radiation sterilization 162 may in particular be carried out anisotropically, with an incidence direction oblique to a normal of the carrier element 146. Even though radiation of the radiation sterilization 162 is intended to penetrate into the interior of the housing 142 in this case, it does not strike the electronics circuit board formed by the carrier element 146 and the electronic components, for example 130 to 136. In this way, radiation damage can be avoided. Furthermore, one or more shielding elements 164 may be provided in the region of the slot 152, for example by the metal housing 144 being bent inward in this region and forming a collar. In this way, radiation entering through the opening of the slot 152 can also be absorbed in the interior of the housing 142 and/or deflected into a harmless direction.

In the exemplary embodiment of the sensor device 110 as represented in FIGS. 2A and 2B, by way of example the carrier 118 of the sensor element 116 and the carrier element 146 are formed as separate elements. This, however, is not necessarily the case. For instance, in the exemplary embodiment represented in FIGS. 2A and 2B, as also optionally in other exemplary embodiments of sensor devices 110 according to the disclosure, at least one carrier 118 of the sensor element 116 and at least one carrier element 146 of the driver 140 may also be fully or partially combined. For example, the carrier 118, which may also be referred to as a sensor abstract, although a plurality of such sensor substrates may be provided (for example in a layer structure), may also form a rigid or flexible circuit board as a carrier element 146 of the driver 140 or of a part of the driver 140. An exemplary embodiment of such an arrangement is represented in FIG. 4. The exemplary embodiment may essentially correspond to the exemplary embodiment represented in FIGS. 2A and 2B, so that reference may be made to the description above for the description of the individual elements. Other configurations, however, are also possible. In this exemplary embodiment, the carrier 118 of the sensor element 116 comprises a flexible circuit board, for example a flex-cable 184, which is at the same time also fed into the housing 142 and used as a carrier element 146 of the driver 140 there. The flex-cable 184 may for example comprise one or more conductor tracks 186 on one or both sides, which may be connected to the electrodes 120 of the sensor element 116 and which can connect the latter to the driver 140. The flex-cable 184 may for example be coated in the region of the sensor element 116 with one or more layers of a cover element 188, for example a cover coating 190, preferably in such a way that the electrodes 120 remain free. The cover coating 190, or the cover element 188, may optionally also form the sealing element 129 or a part thereof at the junction with the housing 142. In particular, the cover element 188 may merge directly and without an interrupting edge into the sealing element 129. The cover element 188 may cover the sensor element 116 fully or partially, and may optionally also cover the housing 142 fully or partially, for example by the cover coating 190 fully or partially covering the housing 142 and in particular the metal housing 144. In this way, the cover element 188 in this exemplary embodiment, or also in other exemplary embodiments, may optionally also be a component of the housing 142 and, for example, provide corrosion protection for the metal housing 144. Furthermore, in this exemplary embodiment or also in other exemplary embodiments, an internal space 192 of the housing 142 may remain free or may be fully or partially filled with a filler 194, for example a dielectric filler 194.

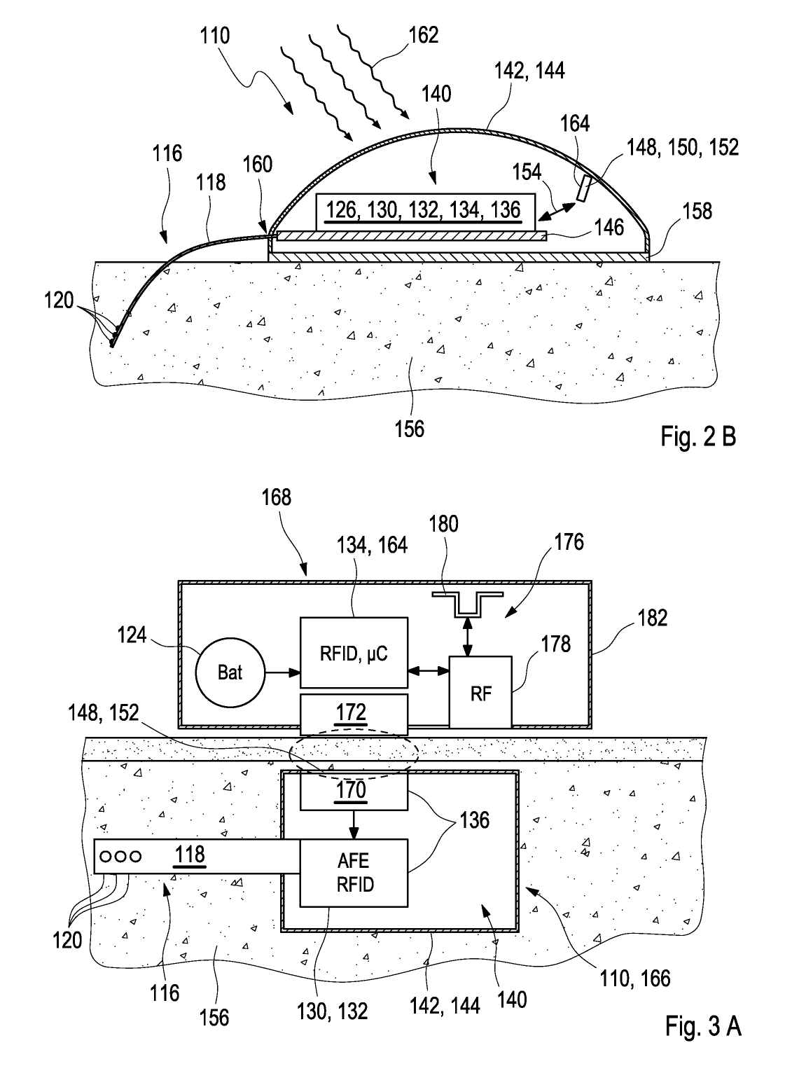

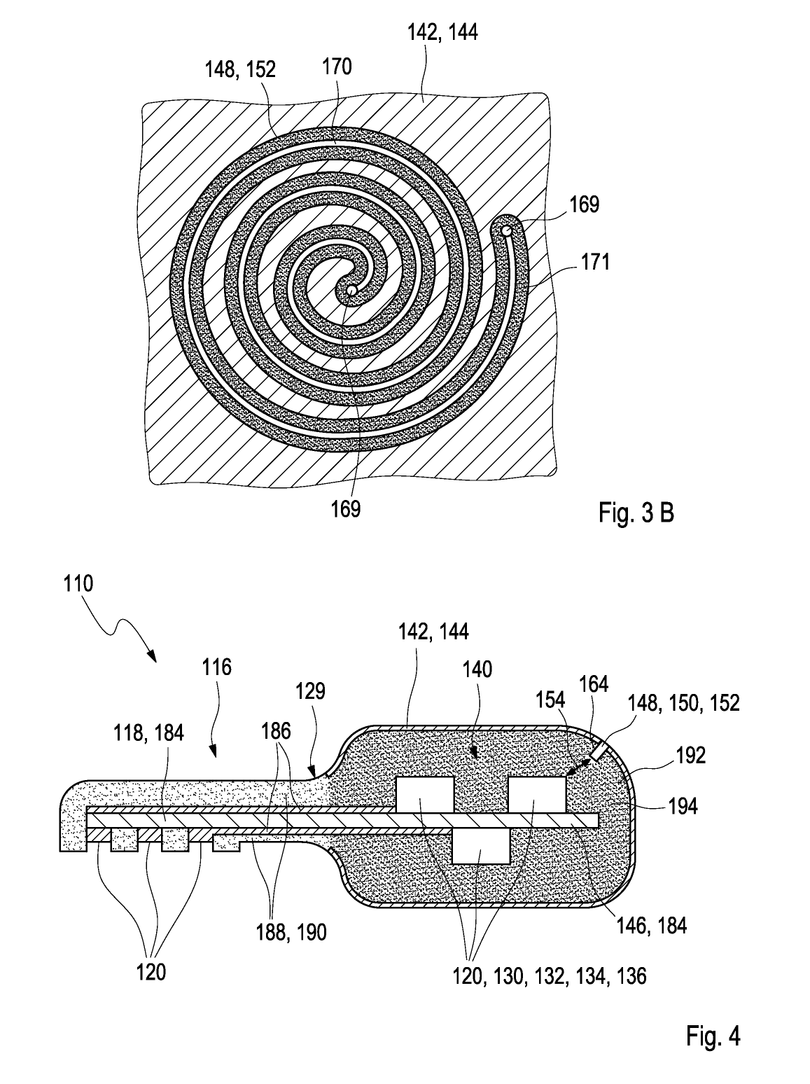

By progressive miniaturization and integration, it is also possible to provide a completely integrated medical device 111, for example a completely integrated sensor device 110, which may also be configured for example as a full implant. This is represented by way of example in FIG. 3A, while FIG. 3B shows a detail of a metal housing 144 of the sensor device according to FIG. 3A in the region of a slot structure 148 in plan view. The sensor device 110 in this case represents a full implant, which can be fully implanted into the body tissue 156. In this case, the sensor device 111 may in principle be configured in a similar way to the sensor device for example according to FIG. 2A. In order to minimize the volume of the driver 140, which is preferable for full implants 166, functionalities of the driver 140 may also be externalized, in this exemplary embodiment by way of example in an external readout apparatus 168. For example, the driver 140 of the sensor device 110 may be reduced merely to an AFE 130, 132. As indicated in FIG. 3A this may additionally have an RFID functionality and therefore already be part of the communication device 136. The communication device 136 may, in a similar way to FIG. 2A, in turn communicate via a slot antenna 150 with at least one external apparatus, for example the readout apparatus 168. As an alternative, however, FIG. 3A represents an option in which the slot structure 148 is used not as a slot antenna 150 but for the purpose of inductive data interchange with the readout apparatus 168. To this end, a coil 170 may be positioned in the slot structure 148 or in the vicinity of the slot structure 148, for example a coil 170 which is incorporated in metal, in which case inductive signals may be interchanged via the coil 170 with a further coil 172 in the readout apparatus 168, through the slot structure 148 of the metal housing 144. The coil 170 may, for example, be configured in the form of a copper wire and may be embedded for example in a dielectric material or filler material 171, for example a sealing polymer which may seal the slot 152. The coil 170 may for example be contacted via terminals 169 and/or penetrations, and for example connected to the communication device 136. The metal housing 144 can be radiation-sterilized during production, without the electronic components, in particular the AFE/RFID 130, 132 suffering radiation damage.

The data-processing device 134 and an energy supply, for example a battery 124, may be externalized into the readout apparatus 168. The readout apparatus 168 may furthermore comprise an RFID reader 164, in order for example to receive the measurement signals of the AFE 130, 132 inductively via the coils 170, 172. In order to supply the full implant 166 permanently with energy and/or transfer data, it is likewise possible to use inductive coupling between the coils 170, 172 according to known RFID technologies.

Furthermore, the readout apparatus 168 may also comprise a communication device 176, for example having a radiofrequency transmitter 178 and an antenna 180. The readout apparatus 168, which need not necessarily be configured in a sterile fashion, may have its own housing 182 which, owing to the preferred arrangement outside the body tissue, need not necessarily be sterilized. Accordingly, the housing 182 may also be made of a nonmetallic material so that conventional antenna structures may be used for the antenna 180, as an alternative or in addition to a slot structure. As an alternative or in addition, however, a slot antenna may preferably also be used in turn for the far-field communication between the readout apparatus 168 and a further apparatus, since under certain circumstances the readout apparatus 168 also has to meet biocompatibility requirements and be sterilizable. In this case, the housing 182 may also in turn be configured fully or partially as a metal housing. Although implanting the antenna 180 for the far-field communication under the skin is also possible in principle, in practice it is difficult to carry out since high frequencies are strongly absorbed in the tissue and since long-wave frequencies would accordingly be required, which would in turn lead to disproportionately large antenna dimensions. The device represented in FIG. 3A, with separation into a readout apparatus 168 and a full implant 166, therefore represents a good compromise since it is possible to carry out near-field communication between the full implant 166 and the readout apparatus 168 inductively, and conventional far-field communication between the readout apparatus 168 and a further apparatus, for example one or more of the further apparatuses mentioned above.

### REFERENCE SYMBOLS

110 sensor device
111 medical device
112 disposable
114 reusable
116 sensor element
117 functional element
118 carrier
120 electrodes
122 electronics part
124 battery
126 memory element
128 plug connector
129 sealing element
130 potentiostat
132 primary amplifier
134 data-processing device
136 communication device
138 base plate
140 driver
142 housing
144 metal housing
146 carrier element
148 slot structure
150 slot antenna
152 Slot
154 excitation device
156 body tissue
158 seal
160 sealing element
162 radiation sterilization
164 shielding elements
166 full implant
168 readout apparatus
169 terminals
170 Coil
171 filler material
172 coil
174 RFID reader
176 communication device
178 radiofrequency sensor
180 antenna, in particular
dipole
182 housing
184 flex-cable
186 conductor track
188 cover element
190 cover coating
192 internal space
194 filler

1. A medical device for carrying out at least one medical function on a human or animal body, wherein the medical device comprises at least one implantable functional element, wherein the medical device further comprises at least one driver having at least one electronic component, wherein the functional element is connectable to the driver, wherein the driver has a housing having at least one metal housing, wherein the metal housing is made entirely of a metallic material, wherein the driver is arranged at least partially in the metal housing, wherein the metal housing fully or at least partially encloses the driver, wherein the driver has at least one wireless communication device, wherein the wireless communication device comprises a device for communication selected from the group consisting of radio communication, communication by inductive coupling and communication via electrical coupling, wherein the metal housing has at least one slot structure, wherein the communication device is adapted to communicate through the slot structure with at least one external apparatus so that the communication takes place by means of the slot structure, wherein the slot structure has at least one metallic shielding collar protruding into the interior of the metal housing as a shielding element.

2. The medical device of claim 1 wherein the medical device comprises a sensor device, wherein the functional element comprises at least one implantable sensor element.

3. The medical device of claim 2 wherein the sensor element comprises at least one electrochemical sensor having at least two sensor electrodes adapted to be arranged in a body tissue in the implanted state of the sensor element, wherein the driver has at least one potentiostat and/or at least one primary amplifier, which is connected to the sensor electrodes, wherein the potentiostat and/or the primary amplifier are arranged in the metal housing.

4. The medical device of claim 1 wherein the metal housing forms a metal shell shielding the driver in at least one direction.

5. The medical device of claim 1 wherein the metal housing is hermetically coupled to a carrier element, wherein the carrier element carries the electronic component.

6. The medical device of claim 1 wherein the slot structure comprises at least one slot antenna.

7. The medical device of claim 6 wherein the communication device comprises at least one excitation device, wherein the excitation device is adapted to excite the slot antenna to emit electromagnetic waves.

8. The medical device of claim 1 wherein the slot structure is at least partially sealed by at least one dielectric material.

9. The medical device of claim 1 wherein the metal housing has a shielding function for electron radiation between 3 and 12 MeV of at least a factor of two.

10. The medical device of claim 1 wherein the metal housing comprises a metal selected from one of the following metals: aluminum, iron, lead, copper, a noble metal, or an alloy.

11. The medical device of claim 1 wherein the slot structure comprises at least one slot, wherein a border of the slot spans a plane, wherein the driver has at least one carrier element, wherein the electronic component is arranged on the carrier element outside a projection of the slot, wherein the projection is a projection perpendicular to the plane onto the carrier element.

12. The medical device of claim 1 wherein the functional element is connected to the driver in a fixed fashion, wherein at least one supply lead of the functional element is fed through at least one sealing element into the housing.

13. The medical device of claim 1 wherein the medical device is a sensor device for monitoring at least one body function.

14. The medical device of claim 1 wherein the implantable functional element is an implantable sensor element.

15. The medical device of claim 9 wherein the metal housing has a shielding function for electron radiation between 3 and 12 MeV of at least a factor of five.

16. The medical device of claim 9 wherein the metal housing has a shielding function for electron radiation between 3 and 12 MeV of at least a factor of ten.

17. The medical device of claim 5 wherein the carrier element is a circuit board.

18. A method for producing a medical device, wherein at least one implantable functional element and at least one driver having at least one electronic component are provided, wherein the functional element is connectable to the driver, wherein the driver is shielded by a housing having at least one metal housing, wherein the metal housing is made entirely of a metallic material, wherein the driver is arranged at least partially in the metal housing, wherein the metal housing fully or partially encloses the driver, wherein the driver has at least one wireless communication device, wherein the wireless communication device comprises a device for communication selected from the group consisting of radio communication, communication by inductive coupling and communication via electrical coupling, wherein the metal housing has at least one slot structure, wherein the communication device is adapted in order to communicate through the slot structure with at least one external apparatus so that the communication takes place by means of the slot structure, wherein the medical device is at least partially sterilized with at least one ionizing radiation, wherein an anisotropic ionizing radiation is used, wherein an incidence direction of the ionizing radiation onto the housing is selected in such a way that ionizing radiation entering into the housing through the slot structure does not reach the electronic component.

19. The method of claim 18 wherein the medical device is a sensor device.

20. The method of claim 18 wherein the medical device is the medical device of claim 1.

21. The method of claim 18 wherein the implantable functional element is an implantable sensor element.

22. The method of claim 18 wherein the anisotropic ionizing radiation is a beta radiation.
